(19) United States
(12) Patent Application Publication
(10) Pub. No.: US 2010/0204690 A1
(43) Pub. Date: Aug. 12, 2010

(54) SINGLE APERTURE ELECTRODE ASSEMBLY

(75) Inventors: Robert Bigley, Redwood City, CA (US); Alana Fulvio, Redwood City, CA (US); Duane W. Marion, Santa Clara, CA (US); Jean Woloszko, Austin, TX (US); Robert H. Dahla, Sunnyvale, CA (US)

Correspondence Address: ARTHROCARE CORPORATION;ATTN: Matthew Scheele, 7500 Rialto Boulevard, Building Two, Suite 100, Austin, TX, 78735-8532, US

(73) Assignee: ARTHROCARE CORPORATION, Austin, TX (US)

(21) Appl. No.: 12/632,551

(22) Filed: Dec. 7, 2009

Related U.S. Application Data
(63) Continuation-in-part of application No. 12/190,752, filed on Aug. 13, 2008.

(51) Int. Cl. A61B 18/14

(52) U.S. Cl. 606/41

(57) ABSTRACT

Systems and methods for securing a screen-type active electrode to the distal tip of an electrosurgical device used for selectively applying electrical energy to a target location within or on a patient's body. A securing electrode is disposed through the screen electrode and mechanically joined to an insulative support body while also creating an electrical connection and mechanical enagement with the screen electrode. The electrosurgical device and related methods are provided for resecting, cutting, partially ablating, aspirating or otherwise removing tissue from a target site, and ablating the tissue in situ. The present methods and systems are particularly useful for removing tissue within joints, e.g., synovial tissue, meniscus, articular cartilage and the like.

### CROSS-REFERENCE TO RELATED APPLICATIONS

This application is a continuation-in-part of U.S. application Ser. No. 12/190,752, filed Aug. 13, 2008, and entitled “Systems and Methods for Screen Electrode Securement,” hereby incorporated herein by reference.

### FIELD OF THE INVENTION

The present invention relates generally to the field of electrosurgery, and more particularly to apparatus and methods for applying high frequency voltage to ablate tissue. More particularly, the present invention relates to apparatus and methods for securing a substantially flat screen-type active electrode to the distal tip of the shaft of an electrosurgical instrument.

### BACKGROUND OF THE INVENTION

Conventional electrosurgical methods are widely used since they generally achieve hemostasis and reduce patient bleeding associated with tissue cutting operations while improving the surgeon's visibility of the treatment area. Many of the electrosurgical devices used in electrosurgery make use of a screen-type active electrode which is typically cut, or etched, from a sheet of conductive material. These electrosurgical devices and procedures, however, suffer from a number of disadvantages. For example, screen-type active electrodes typically require some method of securement to an insulative body and furthermore to the distal tip of the device itself. Failure to adequately secure the screen electrode to the insulative body may result in improper device function and possible patient harm during the electrosurgical procedure.

Prior attempts to secure the screen active electrode to the insulative body have involved mechanical, thermal, and chemical means or various combinations thereof. Numerous mechanical forms of securement have been utilized, while adhesives have been used as a chemical form of joining, and welding the screen may provide one thermal method of joining These mechanical joining methods may also include the use of plastic, or non-recoverable, deformations of the materials being used for securement. However, even in combination with other joining methods, the above-listed methods for fixation provide only marginally effective solutions that typically are challenged over extended periods of use.

Accordingly, devices and methods which allow for the securement of flat screen active electrodes to the insulative body of an electrosurgical instrument while maintaining electrical connections through the insulative body are desired. In particular, mechanical methods for providing reasonable and durable securement of an electrically connected screen active electrode to the insulative body at the distal tip of an electrosurgical device while providing enhanced electrosurgical operating parameters are desired.

### SUMMARY OF THE INVENTION

The present invention provides systems, apparatus and methods for mechanically securing a screen type active electrode to the insulative body at the distal tip of an electrosurgical device. In particular, methods and apparatus are provided for reliably securing the screen electrode over extended periods of use. Further, the methods and systems of the present invention are particularly useful for providing expanded and enhanced electrosurgical operating parameters.

In one aspect of the invention, the method of securement comprises inserting a securing electrode through a channel or slot in both the screen electrode and insulative body. In a configuration where the screen electrode is supported by the insulative body, the securing electrode functions to mechanically couple the screen electrode to the insulative body, and also functions to electrically couple the screen electrode to a high frequency power supply via electrical connectors. The securing electrode may be characterized by extended leg portions having tabs at one end that engage or interfere with the channel in the insulative body, thereby preventing axial movement of the securing electrode. Thus, the securing electrode provides a mechanical method of joining the screen electrode to the insulative body while also providing an electrical connection to transmit RF energy through the insulative body to the screen electrode.

Another configuration of the electrosurgical device according to the present disclosure comprises an active screen electrode having at least two bilateral channels therethrough. At least two bilateral securing electrodes are provided and are respectively inserted through the channels of the screen electrode. Additionally, the device comprises an insulative support member having at least two bilateral channels correspondingly positioned with regard to the screen electrode channels. The bilateral securing electrodes are inserted through the support member and screen electrode channels and may be oriented symmetrically to thereby allow for creation of a zone for RF ablation between the two securing electrodes. Further, the bilateral screen electrodes each have a leg portion with a tab at one end, wherein the tab slides into a locked position within the support member to secure the screen electrode in place.

In certain configurations, the securing electrodes may be characterized by a saw tooth pattern on a superior surface. Additionally, the securing electrodes may be formed in the shape of a staple or bridge, thereby allowing for the creation of another zone of RF ablation in a space between the staple securing electrode and the screen electrode. The added edges formed on the securing electrode in these configurations may result in increased current density and thus promote the formation of improved zones of RF ablation.

In yet another configuration, the active electrode comprises a conductive screen having a single aperture and is positioned over the insulative body at the distal tip of an electrosurgical device in relation to the distal opening of an aspiration lumen. In the representative embodiment, the screen electrode is supported by the insulating support member such that the single aperture on the screen is aligned with the aspiration lumen opening, thereby allowing for the aspiration of unwanted tissue and electrosurgery byproducts from the target site. Additionally, the screen and the distal opening of the aspiration lumen may be positioned on a lateral side of the instrument (i.e., facing 90 degrees from the instrument axis).

In open procedures, the system may further include a fluid delivery element for delivering electrically conducting fluid to the active electrode(s) and the target site. The fluid delivery element may be located on the instrument, e.g., a fluid lumen or tube, or it may be part of a separate instrument. Alternatively, an electrically conducting gel or spray, such as a saline electrolyte or other conductive gel, may be applied to the tissue. In addition, in arthroscopic procedures, the target site will typically already be immersed in a conductive irrigant, i.e., saline. In these embodiments, the apparatus may not have a fluid delivery element. In both embodiments, the electrically conducting fluid will preferably provide a current flow path between the active electrode terminal(s) and the return electrode(s). In an exemplary embodiment, a return electrode is located on the instrument and spaced a sufficient distance from the active electrode terminal(s) to substantially avoid or minimize current shorting therebetween and to isolate the return electrode from tissue at the target site.

In another aspect of the invention, a method comprises positioning one or more active electrode(s) (which may include an active screen electrode and securing electrode) at the target site within a patient's body and applying a suction force to a tissue structure to draw the tissue structure to the active electrode(s). High frequency voltage is then applied between the active electrode(s) and one or more return electrode(s) to ablate the tissue structure. Typically, the tissue structure comprises a flexible or elastic connective tissue, such as synovial tissue. This type of tissue is typically difficult to remove with conventional mechanical and electrosurgery techniques because the tissue moves away from the instrument and/or becomes clogged in the rotating cutting tip of the mechanical shaver or microdebrider. The present invention, by contrast, draws the elastic tissue towards the active electrodes, and then ablates this tissue with the mechanisms described above.

### BRIEF DESCRIPTION OF THE DRAWINGS

FIG. 1 is a perspective view of an electrosurgical system incorporating a power supply and an electrosurgical probe;

FIG. 2 is a perspective view of another electrosurgical system incorporating a power supply, an electrosurgical probe and a supply of electrically conductive fluid for delivering the fluid to the target site;

FIG. 3 is a side view of an electrosurgical probe for ablating and removing tissue;

FIG. 4 is a cross-sectional view of the electrosurgical probe of FIG. 3;

FIG. 5 illustrates a detailed view illustrating ablation of tissue;

FIG. 6 is an enlarged detailed view of the distal end portion of an embodiment of the probe of FIG. 3;

FIGS. 7A and 7B are detailed view of the securing electrode and screen electrode utilized in the electrosurgical probe of FIG. 6;

FIG. 8 is an exploded view of the distal end portion of the probe of FIG. 6;

FIG. 9 is a perspective view of the distal end portion of the probe of FIG. 6;

FIG. 10 is a perspective view of the securing electrodes and screen electrode;

FIG. 11A is a perspective view of a single aperture screen electrode on the distal end portion of an electrosurgical probe in accordance with at least some embodiments;

FIG. 11B is a perspective view of a circular shape aperture screen electrode; and

FIGS. 12A-H illustrate screen electrodes with suction apertures in accordance with at least some embodiments.

### DETAILED DESCRIPTION OF THE INVENTION

The present invention provides systems and methods for selectively applying electrical energy to a target location within or on a patient's body. The present invention is particularly useful in procedures where the tissue site is flooded or submerged with an electrically conducting fluid, such as arthroscopic surgery of the knee, shoulder, ankle, hip, elbow, hand or foot. In other procedures, the present invention may be useful for collagen shrinkage, ablation and/or hemostasis in procedures for treating target tissue alone or in combination with the volumetric removal of tissue. More specifically, the embodiments described herein provide for electrosurgical devices characterized by a substantially flat screen active electrode disposed at the distal tip of the device. Additionally, the present embodiments include apparatus and methods for the mechanical securement of the screen electrode to the insulative body located at the distal tip of the device. Such methods of mechanical securement of the screen electrode may extend the operating period of the electrosurgical device by providing a more secure method of attachment.

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

The electrosurgical device of the present embodiments may have a variety of configurations as described above. However, at least one variation of the embodiments described herein employs a treatment device using Coblation® technology.

As stated above, the assignee of the present invention developed Coblation® technology. Coblation® technology involves the application of a high frequency voltage difference between one or more active electrode(s) and one or more return electrode(s) to develop high electric field intensities in the vicinity of the target tissue. The high electric field intensities may be generated by applying a high frequency voltage that is sufficient to vaporize an electrically conductive fluid over at least a portion of the active electrode(s) in the region between the tip of the active electrode(s) and the target tissue. The electrically conductive fluid may be a liquid or gas, such as isotonic saline, blood, extracelluar or intracellular fluid, delivered to, or already present at, the target site, or a viscous fluid, such as a gel, applied to the target site.

When the conductive fluid is heated enough such that atoms vaporize off the surface faster than they recondense, a gas is formed. When the gas is sufficiently heated such that the atoms collide with each other causing a release of electrons in the process, an ionized gas or plasma is formed (the so-called “fourth state of matter”). Generally speaking, plasmas may be formed by heating a gas and ionizing the gas by driving an electric current through it, or by shining radio waves into the gas. These methods of plasma formation give energy to free electrons in the plasma directly, and then electron-atom collisions liberate more electrons, and the process cascades until the desired degree of ionization is achieved. A more complete description of plasma can be found in Plasma Physics, by R. J. Goldston and P. H. Rutherford of the Plasma Physics Laboratory of Princeton University (1995), the complete disclosure of which is incorporated herein by reference.

As the density of the plasma or vapor layer becomes sufficiently low (i.e., less than approximately 1020 atoms/cm3 for aqueous solutions), the electron mean free path increases to enable subsequently injected electrons to cause impact ionization within the vapor layer. Once the ionic particles in the plasma layer have sufficient energy, they accelerate towards the target tissue. Energy evolved by the energetic electrons (e.g., 3.5 eV to 5 eV) can subsequently bombard a molecule and break its bonds, dissociating a molecule into free radicals, which then combine into final gaseous or liquid species. Often, the electrons carry the electrical current or absorb the radio waves and, therefore, are hotter than the ions. Thus, the electrons, which are carried away from the tissue towards the return electrode, carry most of the plasma's heat with them, allowing the ions to break apart the tissue molecules in a substantially non-thermal manner.

By means of this molecular dissociation (rather than thermal evaporation or carbonization), the target tissue structure is volumetrically removed through molecular disintegration of larger organic molecules into smaller molecules and/or atoms, such as hydrogen, oxygen, oxides of carbon, hydrocarbons and nitrogen compounds. This molecular disintegration completely removes the tissue structure, as opposed to dehydrating the tissue material by the removal of liquid within the cells of the tissue and extracellular fluids, as is typically the case with electrosurgical desiccation and vaporization. A more detailed description of these phenomena can be found in commonly assigned U.S. Pat. No. 5,697,882 the complete disclosure of which is incorporated herein by reference.

In some applications of the Coblation® technology, high frequency (RF) electrical energy is applied in an electrically conducting media environment to shrink or remove (i.e., resect, cut, or ablate) a tissue structure and to seal transected vessels within the region of the target tissue. Coblation® technology is also useful for sealing larger arterial vessels, e.g., on the order of about 1 mm in diameter. In such applications, a high frequency power supply is provided having an ablation mode, wherein a first voltage is applied to an active electrode sufficient to effect molecular dissociation or disintegration of the tissue, and a coagulation mode, wherein a second, lower voltage is applied to an active electrode (either the same or a different electrode) sufficient to heat, shrink, and/or achieve hemostasis of severed vessels within the tissue.

The amount of energy produced by the Coblation® device may be varied by adjusting a variety of factors, such as: the number of active electrodes; electrode size and spacing; electrode surface area; asperities and sharp edges on the electrode surfaces; electrode materials; applied voltage and power; current limiting means, such as inductors; electrical conductivity of the fluid in contact with the electrodes; density of the fluid; and other factors. Accordingly, these factors can be manipulated to control the energy level of the excited electrons. Since different tissue structures have different molecular bonds, the Coblation® device may be configured to produce energy sufficient to break the molecular bonds of certain tissue but insufficient to break the molecular bonds of other tissue. For example, fatty tissue (e.g., adipose) has double bonds that require an energy level substantially higher than 4 eV to 5 eV (typically on the order of about 8 eV) to break. Accordingly, the Coblation® technology generally does not ablate or remove such fatty tissue; however, it may be used to effectively ablate cells to release the inner fat content in a liquid form. Of course, factors may be changed such that these double bonds can also be broken in a similar fashion as the single bonds (e.g., increasing voltage or changing the electrode configuration to increase the current density at the electrode tips). A more complete description of these phenomena can be found in commonly assigned U.S. Pat. Nos. 6,355,032, 6,149,120 and 6,296,136, the complete disclosures of which are incorporated herein by reference.

The active electrode(s) of a Coblation® device may be supported within or by an inorganic insulating support member positioned near the distal end of the instrument shaft. The return electrode may be located on the instrument shaft, on another instrument or on the external surface of the patient (i.e., a dispersive pad). The proximal end of the instrument(s) will include the appropriate electrical connections for coupling the return electrode(s) and the active electrode(s) to a high frequency power supply, such as an electrosurgical generator.

Further discussion of applications and devices using Coblation® technology may be found as follows. Issued U.S. Pat. Nos. 6,296,638; and 7,241,293 both of which are incorporated by reference. Pending U.S. application Ser. No. 11/612,995 filed Dec. 19, 2006, which is incorporated by reference.

In one example of a Coblation® device for use with the presently-described embodiments, the return electrode of the device is typically spaced proximally from the active electrode(s) a suitable distance to avoid electrical shorting between the active and return electrodes in the presence of electrically conductive fluid. In many cases, the distal edge of the exposed surface of the return electrode is spaced about 0.5 mm to 25 mm from the proximal edge of the exposed surface of the active electrode(s), preferably about 1.0 mm to 5.0 mm. Of course, this distance may vary with different voltage ranges, conductive fluids, and depending on the proximity of tissue structures to active and return electrodes. The return electrode will typically have an exposed length in the range of about 1 mm to 20 mm.

A Coblation® treatment device for use according to the present descriptions may use a single active electrode or an array of active electrodes spaced around the distal surface of a catheter or probe. In the latter embodiment, the electrode array usually includes a plurality of independently current-limited and/or power-controlled active electrodes to apply electrical energy selectively to the target tissue while limiting the unwanted application of electrical energy to the surrounding tissue and environment resulting from power dissipation into surrounding electrically conductive fluids, such as blood, normal saline, and the like. The active electrodes may be independently current-limited by isolating the terminals from each other and connecting each terminal to a separate power source that is isolated from the other active electrodes. Alternatively, the active electrodes may be connected to each other at either the proximal or distal ends of the catheter to form a single wire that couples to a power source.

In certain configurations, each individual active electrode in the electrode array may be electrically insulated from all other active electrodes in the array within the instrument and is connected to a power source which is isolated from each of the other active electrodes in the array or to circuitry which limits or interrupts current flow to the active electrode when low resistivity material (e.g., blood, electrically conductive saline irrigant or electrically conductive gel) causes a lower impedance path between the return electrode and the individual active electrode. The isolated power sources for each individual active electrode may be separate power supply circuits having internal impedance characteristics which limit power to the associated active electrode when a low impedance return path is encountered. By way of example, the isolated power source may be a user selectable constant current source. In this embodiment, lower impedance paths will automatically result in lower resistive heating levels since the heating is proportional to the square of the operating current times the impedance. Alternatively, a single power source may be connected to each of the active electrodes through independently actuatable switches, or by independent current limiting elements, such as inductors, capacitors, resistors and/or combinations thereof. The current limiting elements may be provided in the instrument, connectors, cable, controller, or along the conductive path from the controller to the distal tip of the instrument. Alternatively, the resistance and/or capacitance may occur on the surface of the active electrode(s) due to oxide layers which form selected active electrodes (e.g., titanium or a resistive coating on the surface of metal, such as platinum).

The Coblation® device is not limited to electrically isolated active electrodes, or even to a plurality of active electrodes. For example, in certain embodiments the array of active electrodes may be connected to a single lead that extends through the catheter shaft to a power source of high frequency current.

The voltage difference applied between the return electrode(s) and the active electrode(s) will be at high or radio frequency, typically between about 5 kHz and 20 MHz, usually being between about 30 kHz and 2.5 MHz, preferably being between about 50 kHz and 500 kHz, often less than 350 kHz, and often between about 100 kHz and 200 kHz. In some applications, applicant has found that a frequency of about 100 kHz is useful because the tissue impedance is much greater at this frequency. In other applications, such as procedures in or around the heart or head and neck, higher frequencies may be desirable (e.g., 400-600 kHz) to minimize low frequency current flow into the heart or the nerves of the head and neck.

The RMS (root mean square) voltage applied will usually be in the range from about 5 volts to 1000 volts, preferably being in the range from about 10 volts to 500 volts, often between about 150 volts to 400 volts depending on the active electrode size, the operating frequency and the operation mode of the particular procedure or desired effect on the tissue (i.e., contraction, coagulation, cutting or ablation).

Typically, the peak-to-peak voltage for ablation or cutting with a square wave form will be in the range of 10 volts to 2000 volts and preferably in the range of 100 volts to 1800 volts and more preferably in the range of about 300 volts to 1500 volts, often in the range of about 300 volts to 800 volts peak to peak (again, depending on the electrode size, number of electrons, the operating frequency and the operation mode). Lower peak-to-peak voltages will be used for tissue coagulation, thermal heating of tissue, or collagen contraction and will typically be in the range from 50 to 1500, preferably 100 to 1000 and more preferably 120 to 400 volts peak-to-peak (again, these values are computed using a square wave form). Higher peak-to-peak voltages, e.g., greater than about 800 volts peak-to-peak, may be desirable for ablation of harder material, such as bone, depending on other factors, such as the electrode geometries and the composition of the conductive fluid.

As discussed above, the voltage is usually delivered in a series of voltage pulses or alternating current of time varying voltage amplitude with a sufficiently high frequency (e.g., on the order of 5 kHz to 20 MHz) such that the voltage is effectively applied continuously (as compared with, e.g., lasers claiming small depths of necrosis, which are generally pulsed about 10 Hz to 20 Hz). In addition, the duty cycle (i.e., cumulative time in any one-second interval that energy is applied) is on the order of about 50% for the present invention, as compared with pulsed lasers which typically have a duty cycle of about 0.0001%.

The preferred power source of the present invention delivers a high frequency current selectable to generate average power levels ranging from several milliwatts to tens of watts per electrode, depending on the volume of target tissue being treated, and/or the maximum allowed temperature selected for the instrument tip. The power source allows the user to select the voltage level according to the specific requirements of a particular neurosurgery procedure, cardiac surgery, arthroscopic surgery, dermatological procedure, ophthalmic procedures, open surgery or other endoscopic surgery procedure. For cardiac procedures and potentially for neurosurgery, the power source may have an additional filter, for filtering leakage voltages at frequencies below 100 kHz, particularly voltages around 60 kHz. Alternatively, a power source having a higher operating frequency, e.g., 300 kHz to 600 kHz may be used in certain procedures in which stray low frequency currents may be problematic. A description of one suitable power source can be found in commonly assigned U.S. Pat. Nos. 6,142,992 and 6,235,020, the complete disclosure of both patents are incorporated herein by reference for all purposes.

The power source may be current limited or otherwise controlled so that undesired heating of the target tissue or surrounding (non-target) tissue does not occur. In a presently preferred embodiment of the present invention, current limiting inductors are placed in series with each independent active electrode, where the inductance of the inductor is in the range of 10 uH to 50,000 uH, depending on the electrical properties of the target tissue, the desired tissue heating rate and the operating frequency. Alternatively, capacitor-inductor (LC) circuit structures may be employed, as described previously in U.S. Pat. No. 5,697,909, the complete disclosure of which is incorporated herein by reference. Additionally, current-limiting resistors may be selected. Preferably, these resistors will have a large positive temperature coefficient of resistance so that, as the current level begins to rise for any individual active electrode in contact with a low resistance medium (e.g., saline irrigant or blood), the resistance of the current limiting resistor increases significantly, thereby minimizing the power delivery from said active electrode into the low resistance medium (e.g., saline irrigant or blood).

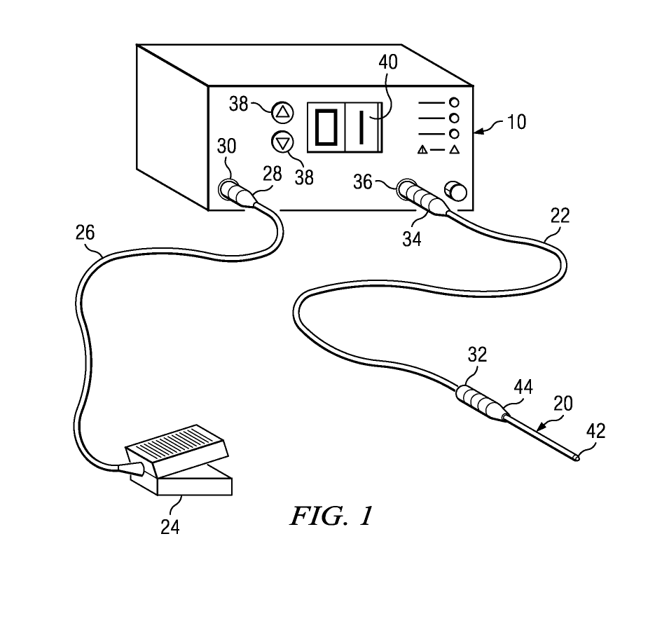

Referring now to FIG. 1, an exemplary electrosurgical system for resection, ablation, coagulation and/or contraction of tissue will now be described in detail. As shown, certain embodiments of the electrosurgical system generally include an electrosurgical probe 20 connected to a power supply 10 for providing high frequency voltage to one or more electrode terminals on probe 20. Probe 20 includes a connector housing 44 at its proximal end, which can be removably connected to a probe receptacle 32 of a probe cable 22. The proximal portion of cable 22 has a connector 34 to couple probe 20 to power supply 10 at receptacle 36. Power supply 10 has an operator controllable voltage level adjustment 38 to change the applied voltage level, which is observable at a voltage level display 40. Power supply 10 also includes one or more foot pedals 24 and a cable 26 which is removably coupled to a receptacle 30 with a cable connector 28. The foot pedal 24 may also include a second pedal (not shown) for remotely adjusting the energy level applied to electrode terminals 42, and a third pedal (also not shown) for switching between an ablation mode and a coagulation mode.

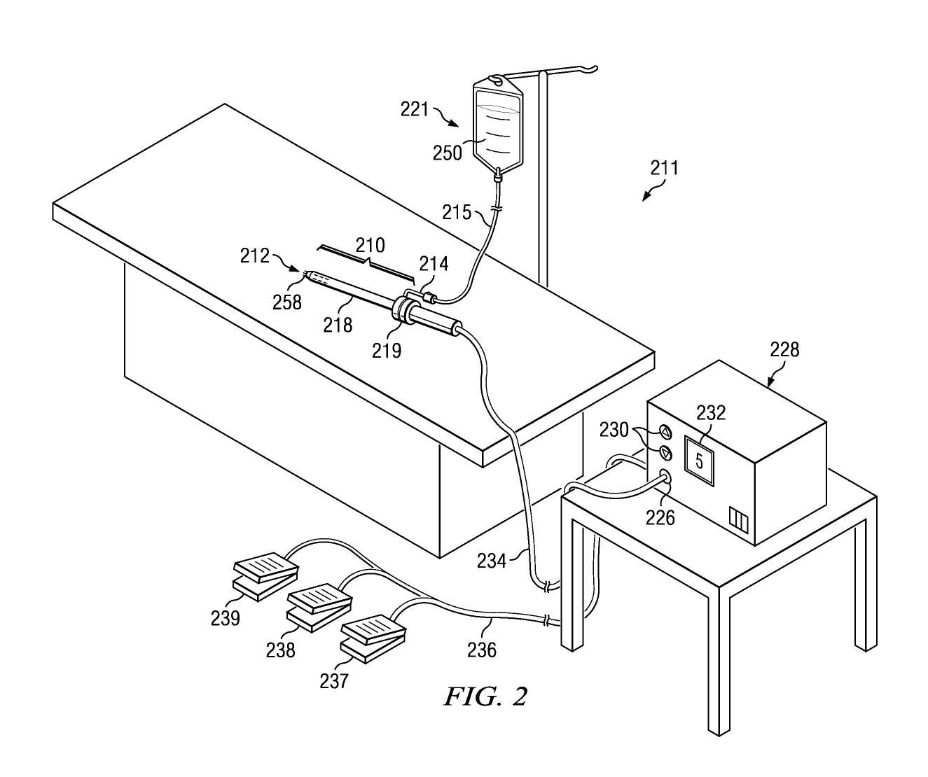

Referring now to FIG. 2, an exemplary electrosurgical system 211 for treatment of tissue in ‘dry fields’ will now be described in detail. Of course, system 211 may also be used in ‘wet field’, i.e., the target site is immersed in electrically conductive fluid. However, this system is particularly useful in ‘dry fields’ where the fluid is preferably delivered through electrosurgical probe to the target site. As shown, electrosurgical system 211 generally comprises an electrosurgical handpiece or probe 210 connected to a power supply 228 for providing high frequency voltage to a target site and a fluid source 221 for supplying electrically conducting fluid 250 to probe 210. The system 211 may also include a vacuum source (not shown) for coupling to a suction lumen disposed in probe 210 (not shown) via a connection tube (not shown) on probe 210 for aspirating the target site, as discussed below in more detail.

As shown, probe 210 generally includes a proximal handle 219 and an elongate shaft 218 having an array 212 of electrode terminals 258 at its distal end. A connecting cable 234 has a connector 226 for electrically coupling the electrode terminals 258 to power supply 228. The electrode terminals 258 are electrically isolated from each other and each of the terminals 258 is connected to an active or passive control network within power supply 228 by means of a plurality of individually insulated conductors (not shown). A fluid supply tube 215 is connected to a fluid tube 214 of probe 210 for supplying electrically conducting fluid 250 to the target site.

Similar to the above embodiment shown in FIG. 1, power supply 228 has an operator controllable voltage level adjustment 230 to change the applied voltage level, which is observable at a voltage level display 232. Power supply 228 also includes first, second and third foot pedals 237, 238, 239 and a cable 236 which is removably coupled to power supply 228. The foot pedals 237, 238, 239 allow the surgeon to remotely adjust the energy level applied to electrode terminals 258. In an exemplary embodiment, first foot pedal 237 is used to place the power supply into the “ablation” mode and second foot pedal 238 places power supply 228 into the “coagulation” mode. The third foot pedal 239 allows the user to adjust the voltage level within the “ablation” mode. In the ablation mode, a sufficient voltage is applied to the electrode terminals to establish the requisite conditions for molecular dissociation of the tissue (i.e., vaporizing a portion of the electrically conductive fluid, ionizing charged particles within the vapor layer and accelerating these charged particles against the tissue). As discussed above, the requisite voltage level for ablation will vary depending on the number, size, shape and spacing of the electrodes, the distance in which the electrodes extend from the support member, etc. Once the surgeon places the power supply in the “ablation” mode, voltage level adjustment 230 or third foot pedal 239 may be used to adjust the voltage level to adjust the degree or aggressiveness of the ablation.

It will be recognized that the voltage and modality of the power supply may be controlled by other input devices. However, applicant has found that foot pedals are convenient methods of controlling the power supply while manipulating the probe during a surgical procedure.

In the coagulation mode, the power supply 228 applies a low enough voltage to the electrode terminals (or the coagulation electrode) to avoid vaporization of the electrically conductive fluid and subsequent molecular dissociation of the tissue. The surgeon may automatically toggle the power supply between the ablation and coagulation modes by alternatively stepping on foot pedals 237, 238, respectively. This allows the surgeon to quickly move between coagulation and ablation in situ, without having to remove his/her concentration from the surgical field or without having to request an assistant to switch the power supply. By way of example, as the surgeon is sculpting soft tissue in the ablation mode, the probe typically will simultaneously seal and/or coagulation small severed vessels within the tissue. However, larger vessels, or vessels with high fluid pressures (e.g., arterial vessels) may not be sealed in the ablation mode. Accordingly, the surgeon can simply step on foot pedal 238, automatically lowering the voltage level below the threshold level for ablation, and apply sufficient pressure onto the severed vessel for a sufficient period of time to seal and/or coagulate the vessel. After this is completed, the surgeon may quickly move back into the ablation mode by stepping on foot pedal 237.

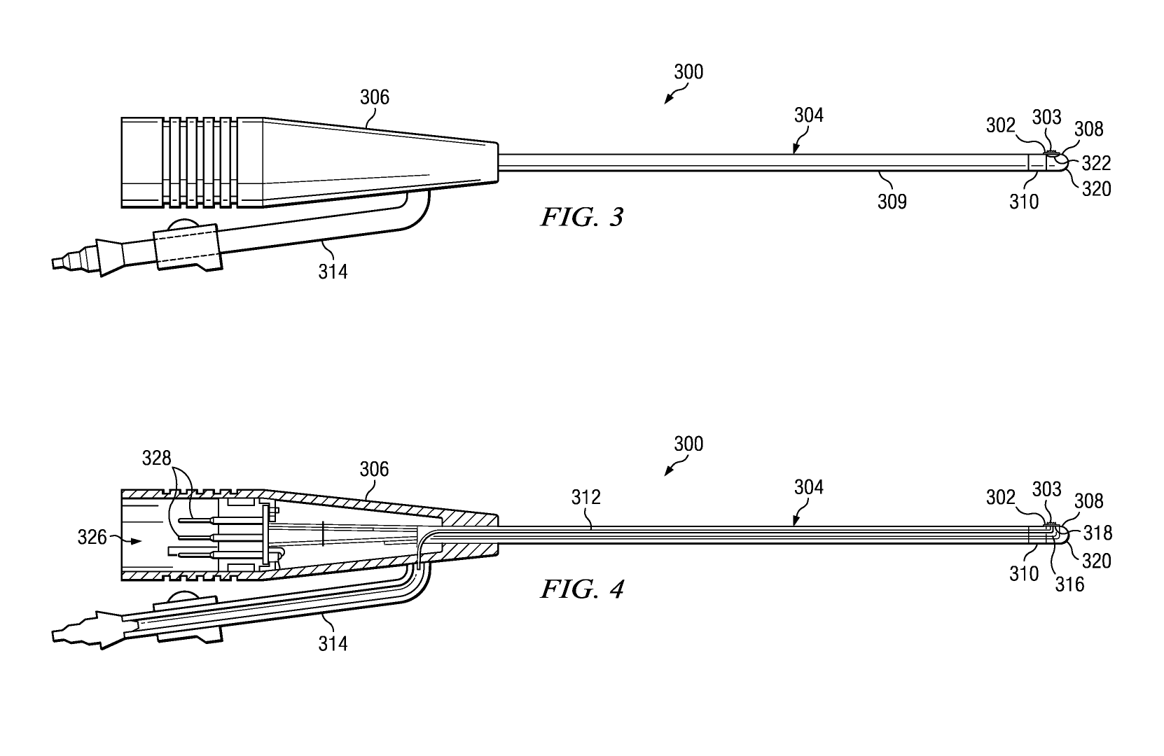

Now referring to FIGS. 3 and 4, an exemplary electrosurgical probe 300 incorporating an active screen electrode 302 is illustrated. Probe 300 may include an elongated shaft 304 which may be flexible or rigid, a handle 306 coupled to the proximal end of shaft 304 and an electrode support member 308 coupled to the distal end of shaft 304. Probe 300 further includes active screen electrode 302 and securing electrode 303. Return electrode 310 is spaced proximally from screen electrode 302 and provides a method for completing the current path between screen electrode 302 and securing electrode 303. As shown, return electrode 310 preferably comprises an annular exposed region of shaft 304 slightly proximal of insulative support member 308, typically about 0.5 to 10 mm and more preferably about 1 to 10 mm. Securing electrode 303 and return electrode 310 are each coupled to respective connectors 328 disposed in handle 306 (as illustrated in FIG. 4) that extend to the proximal end of probe 300, where connectors 328 are suitably electrically connected to a power supply (e.g., power supply 10 in FIG. 1 or power supply 228 in FIG. 2). As shown in FIG. 4, handle 306 defines an inner cavity 326 that houses the electrical connectors 328, and provides a suitable interface for connection to an electrical connecting cable (e.g., cable 22 in FIG. 1 or cable 234 in FIG. 2).

Still referencing FIGS. 3 and 4, in certain embodiments screen electrode 302, securing electrode 303 and insulative support member 308 are configured such that screen electrode 302 and securing electrode 303 are positioned on a lateral side of the shaft 304 (e.g., 90 degrees from the shaft axis) to allow the physician to access tissue that is offset from the axis of the portal or arthroscopic opening into the joint cavity in which the shaft 304 passes during the procedure. To accomplish this, probe 300 may include an electrically insulating cap 320 coupled to the distal end of shaft 304 and having a lateral opening 322 for receiving support member 308, screen electrode 302, and securing electrode 303.

Shaft 304 preferably comprises an electrically conducting material, usually metal, which is selected from the group consisting of tungsten, stainless steel alloys, platinum or its alloys, titanium or its alloys, molybdenum or its alloys, and nickel or its alloys. Shaft 304 may include an electrically insulating jacket 309, which is typically formed as one or more electrically insulating sheaths or coatings, such as polytetrafluoroethylene, polyimide, and the like. The provision of the electrically insulating jacket over the shaft prevents direct electrical contact between these metal elements and any adjacent body structure or the surgeon. Such direct electrical contact between a body structure and an exposed electrode could result in unwanted heating and necrosis of the structure at the point of contact causing necrosis.

The probe 300 further includes a suction connection tube 314 for coupling to a source of vacuum, and an inner suction lumen 312 (FIG. 4) for aspirating excess fluids, tissue fragments, and/or products of ablation (e.g., bubbles) from the target site. Preferably, connection tube 314 and suction lumen 312 are fluidly connected, thereby providing the ability to create a suction pressure in lumen 312 that allows the surgeon to draw loose tissue, e.g., synovial tissue, towards the screen electrode 302. Typically, the vacuum source is a standard hospital pump that provides suction pressure to connection tube 314 and lumen 312. As shown in FIGS. 3 and 4, internal suction lumen 312, which preferably comprises peek tubing, extends from connection tube 314 in handle 306, through shaft 304 to an axial opening 316 in support member 308, through support member 308 to a lateral opening 318 in support member 308. Lateral opening 318 is positioned adjacent to screen electrode 302, which further includes a suction port (not shown) disposed on the surface of screen electrode 302 and fluidly connected to lateral opening 318 for allowing aspiration therethrough, as discussed below in more detail.

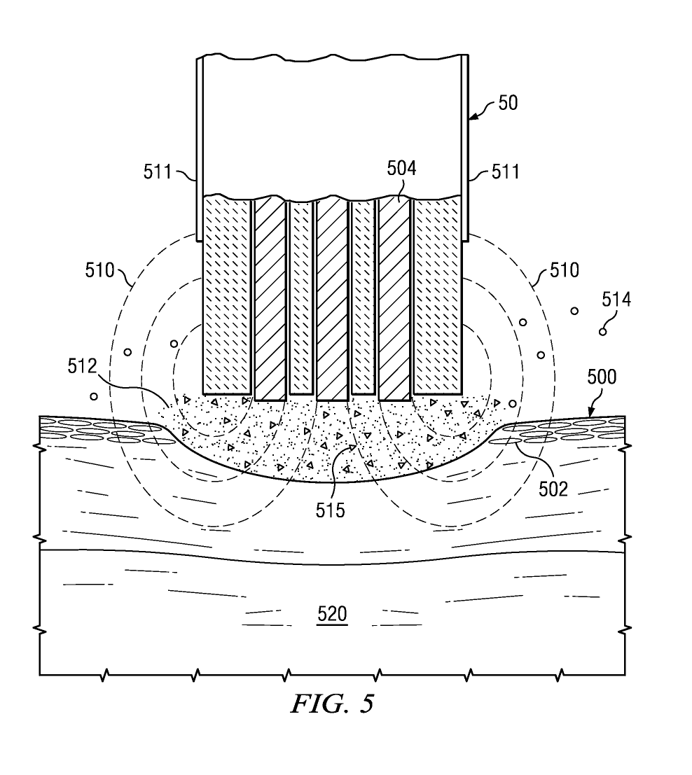

FIG. 5 representatively illustrates in more detail the removal of a target tissue by use of an embodiment of electrosurgical probe 50 according to the present disclosure. As shown, the high frequency voltage is sufficient to convert the electrically conductive fluid (not shown) between the target tissue 502 and active electrode terminal(s) 504 into an ionized vapor layer 512 or plasma. As a result of the applied voltage difference between electrode terminal(s) 504 and the target tissue 502 (i.e., the voltage gradient across the plasma layer 512), charged particles 515 in the plasma are accelerated. At sufficiently high voltage differences, these charged particles 515 gain sufficient energy to cause dissociation of the molecular bonds within tissue structures in contact with the plasma field. This molecular dissociation is accompanied by the volumetric removal (i.e., ablative sublimation) of tissue and the production of low molecular weight gases 514, such as oxygen, nitrogen, carbon dioxide, hydrogen and methane. The short range of the accelerated charged particles 515 within the tissue confines the molecular dissociation process to the surface layer to minimize damage and necrosis to the underlying tissue 520.

During the process, the gases 514 will be aspirated through a suction opening and suction lumen to a vacuum source (not shown). In addition, excess electrically conductive fluid, and other fluids (e.g., blood) will be aspirated from the target site 500 to facilitate the surgeon's view. During ablation of the tissue, the residual heat generated by the current flux lines 510 (typically less than 150° C.) between electrode terminals 504 and return electrode 511 will usually be sufficient to coagulate any severed blood vessels at the site. If not, the surgeon may switch the power supply (not shown) into the coagulation mode by lowering the voltage to a level below the threshold for fluid vaporization, as discussed above. This simultaneous hemostasis results in less bleeding and facilitates the surgeon's ability to perform the procedure. Once the blockage has been removed, aeration and drainage are reestablished to allow the sinuses to heal and return to their normal function.

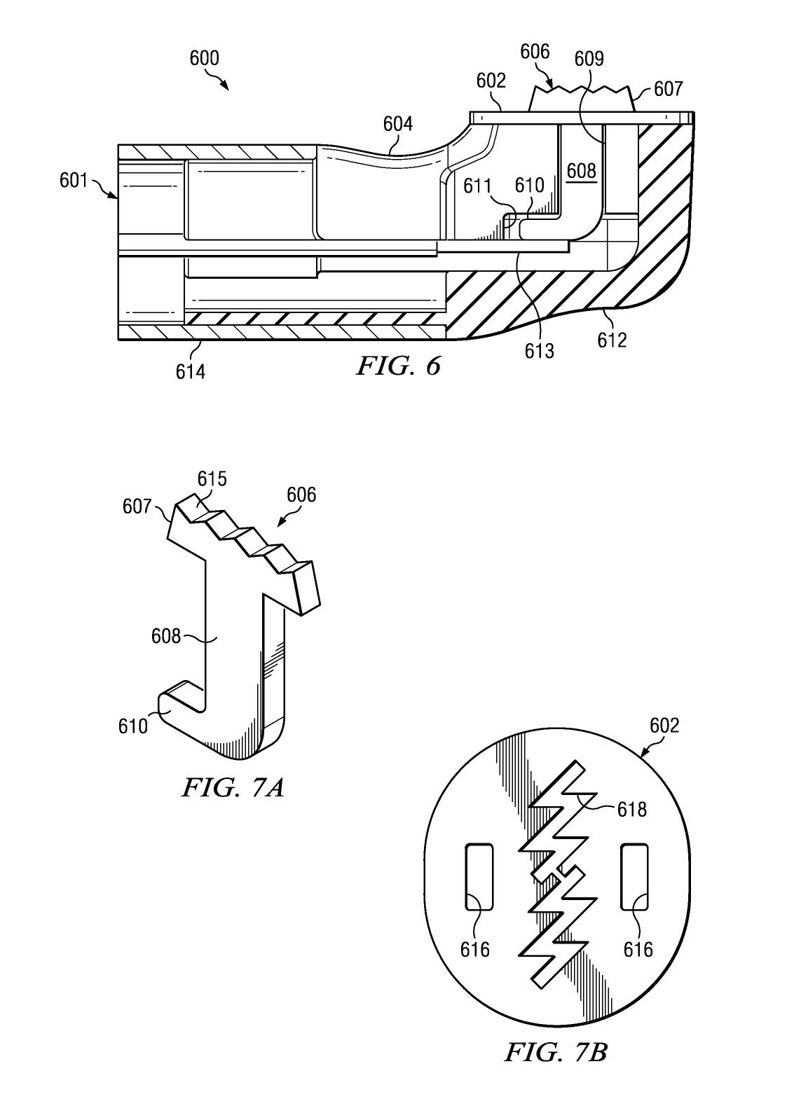

Now referring to FIG. 6, the distal end portion of a preferred embodiment of an electrosurgical probe according to present disclosure is shown. Electrosurgical probe 600 comprises active screen electrode 602 mounted to insulative support member 604 disposed at a distal end of elongate shaft 601. Probe 600 also includes electrically insulating cap 612 coupled to the end of shaft 601 and configured to receive screen electrode 602 and support member 604. In preferred embodiments, securing electrode 606 extends through screen electrode 602 and support member 604 to mechanically secure screen electrode 602 to support member 604 and electrically insulating cap 612. In certain configurations, securing electrodes 606 may be characterized by head 607, leg 608, and tab 610. Preferably, head 607 contacts or engages the superior surface of screen electrode 602, thereby providing an electrical means for the transmission of RF energy between securing electrode 606 and screen electrode 602. Support member 604 may be characterized by channel 609 and slot 611, wherein channel 609 is oriented perpendicularly with respect to the axis of shaft 601 and slot 611 is oriented axially with respect to the axis of shaft 601. Wire 613 extends proximally from slot 611, and is electrically connected to the electrical connectors disposed in the handle of the probe (as discussed above). Return electrode 614 is spaced proximally from screen electrode 602. As discussed above, in this embodiment screen electrode 602 and support member 604 are configured such that screen electrode 602 is positioned on the lateral side of shaft 601 (e.g., 90 degrees from the shaft axis) to allow the physician to access tissue that is offset from the axis of the port or arthroscopic opening into the joint cavity in which shaft 601 passes during the procedure.

Referring now to FIG. 7A, an embodiment of securing electrode 606 is shown. Securing electrode 606 may be formed with a conductive material such as tungsten, and the shape and profile of securing electrode 606 may be manufactured via etching, laser cutting, or injection molding. In certain configurations, securing electrode 606 may be characterized by saw tooth pattern 615 on the superior plasma forming surface of securing electrode 606. The added edges formed on securing electrodes 606 by saw tooth pattern 615 in this configuration may result in increased current density and thus promote the formation of improved zones for plasma formation and RF ablation.

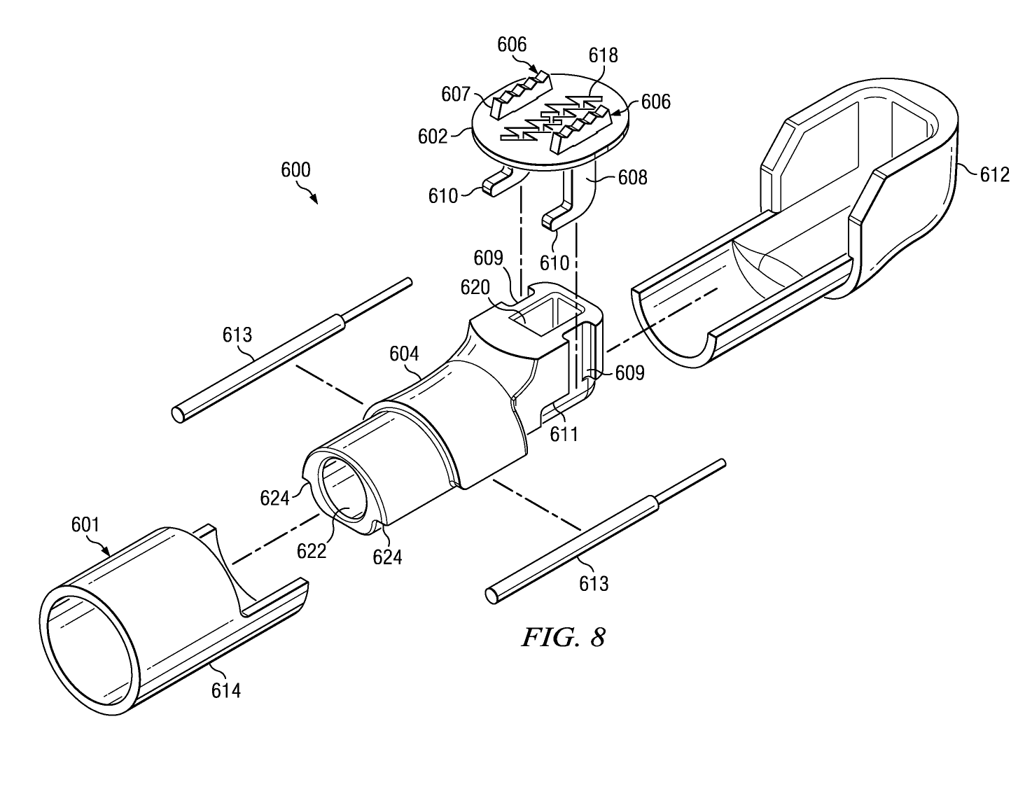

Referring now to FIG. 7B, screen electrode 602 will comprise a conductive material, such as tungsten, titanium, molybdenum, stainless steel, aluminum, gold, copper or the like. Screen electrode 602 will usually have a diameter in the range of about 0.5 to 8 mm, preferably about 1 to 4 mm, and a thickness of about 0.05 to about 2.5 mm, preferably about 0.1 to 1 mm. Screen electrode 602 may have a variety of different shapes, such as the shape shown in FIG. 7B. Screen electrode may have slots 616 therethrough, and may comprise suction opening 618 having sizes and configurations that may vary depending on the particular application. The exposed surface of screen electrode 602 is preferably generally planar, with no projections extending from the surface of screen electrode 602 or from an area associated with suction opening 618. Suction opening 618 will typically be large enough to allow ablated tissue fragments to pass through into suction lumen port 620 (see FIG. 8), typically being about 2 to 30 mils in diameter, preferably about 5 to 20 mils in diameter. In some applications, it may be desirable to only aspirate fluid and the gaseous products of ablation (e.g., bubbles) so that the holes may be much smaller, e.g., on the order of less than 10 mils, often less than 5 mils. In certain configurations, suction opening 618 may be formed in the shape of a zigzag or lightning bolt.

Suction opening 618 is preferably formed in a design such as a zigzag or lightning bolt shape that affords for increased edge surface exposure around along the boundary of suction opening 618 in combination with a sufficiently opening area to allow material desired to be aspirated to enter the suction lumen via the suction lumen port. Consistent with any variation of selected aperture shape, suction opening 618 is characterized by an opening perimeter 619 and an opening area 620. Opening perimeter 619 may be the sum of the length of the exposed edge surfaces bounding suction opening 618, and opening area 620 may be the two-dimensional size of the region bounded by the closed opening perimeter 619. Alternatively, the opening area 620 may be the total area of the exposed surface of a projected three-dimensional solid corresponding in shape to that of suction opening 618. As is discussed below in more detail, it is preferred that the ratio of opening perimeter 619 to opening area 620 be greater than the ratio of a corresponding circular opening perimeter to a circular opening area where the suction opening is formed in a generally circular shape.

Referring now to FIG. 8, insulative electrode support member 604 preferably comprises an inorganic material, such as glass, ceramic, silicon nitride, alumina or the like, that has been formed with lateral and axial suction lumen openings 620, 622, and with one or more lateral axial passages 624 for receiving electrical wires 613. Wires 613 extend from electrical connectors (i.e., electrical connectors 328 in FIG. 4), through shaft 601 and passages 624 in support member 604, terminating in proximity to slots 611 and tabs 610 of securing electrodes 606. Wires 613 are electrically connected to securing electrodes 606 (e.g., by a laser welding process) thereby electrically coupling securing electrodes 606 and screen electrode 602 to a high frequency power supply. Referring to FIGS. 6, 7B, and 8, legs 608 may extend through slots 616 of screen electrode 602 and channels 609 of support member 604, and tabs 610 may be inserted into slots 611 of support member 604 such that tabs 610 interfere or engage with a portion of support member 604. The placement of securing electrodes 606 such that tabs 610 are inserted into slots 611 creates a mechanical method of joining securing electrodes 606 to support member 604 and thereby prevents securing electrodes 606 from moving axially with respect to shaft 601 and support member 604. Additionally, the method of mechanical securement results in the capture of screen electrode 602 between securing electrodes 606 and support member 604. Further, as described above the contact between heads 607 of securing electrodes 606 and screen electrode 602 provides a method to electrically transmit RF energy through support member 604 to screen electrode 602.

In additional embodiments, the mechanical method of joining may comprise complementary helical threads cut in channels 609 of support member 604 and respectively in legs 608 of securing electrodes 606, wherein legs 608 of securing electrodes 606 are operable to threadingly engage channels 609 of support member 604. Additional embodiments of the present disclosure may include configurations where tabs 610 are formed in a barb or arrowhead shape and are disposed in interference with support member 604. Moreover, in additional embodiments tabs 610 may be completely enclosed within support member 604, and may be further secured to support member 604 by epoxy.

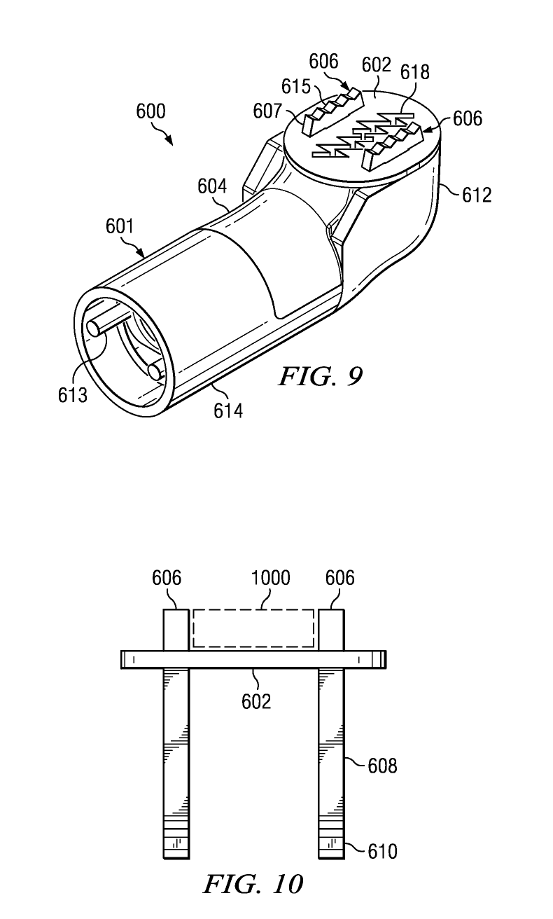

Referring now to FIGS. 9 and 10, the distal end portion of representative probe 600 is shown with at least two bilateral securing electrodes 606 thereon. In this configuration, securing electrodes 606 may be oriented symmetrically about the central axis of shaft 601, and may thereby allow for creation of a zone for RF ablation or plasma chamber 1000 between the symmetrically oriented bilateral securing electrodes 606 as well as between securing electrodes 606 and screen electrode 602 (see i.e., FIG. 10). Incorporation of symmetrical securing electrodes 606 may allow for the creation of a three dimensional zone represented by plasma zone 1000 for carrying out RF ablation.

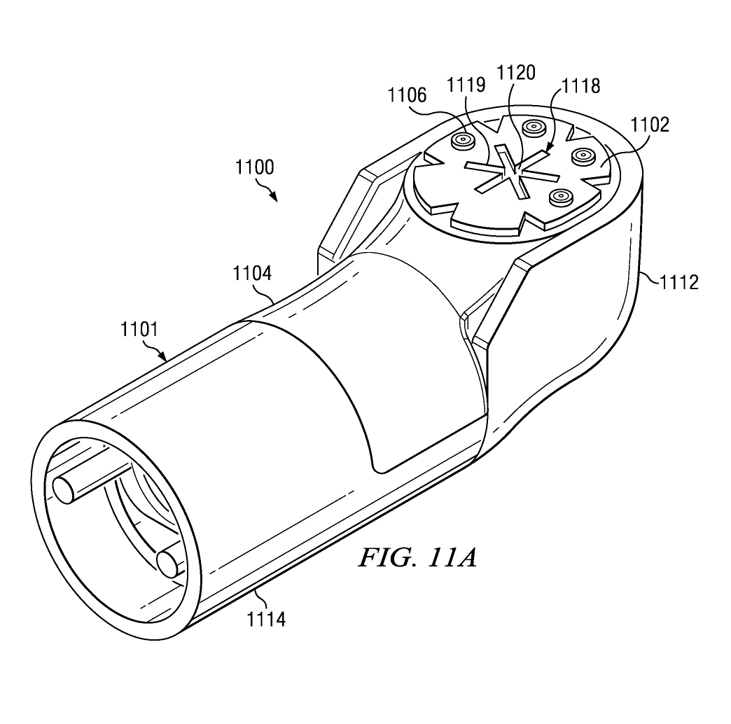

Referring now to FIG. 11A, an alternative screen electrode configuration is shown in accordance with at least some embodiments. Electrosurgical probe 1100 comprises active screen electrode 1102 mounted to insulative support member 1104 disposed at a distal end of elongate shaft 1101. Probe 1100 also includes electrically insulating cap 1112 coupled to the end of shaft 1101 and configured to receive screen electrode 1102 and support member 1104. In certain embodiments, at least one securing electrode 1106 extends through screen electrode 1102 and support member 1104 to mechanically secure screen electrode 1102 to support member 1104 and electrically insulating cap 1112. Return electrode 1114 is spaced proximally from screen electrode 1102. As discussed above, in this embodiment screen electrode 1102 and support member 1104 are configured such that screen electrode 1102 is positioned on the lateral side of shaft 1101 (e.g., 90 degrees from the shaft axis) to allow the physician to access tissue that is offset from the axis of the port or arthroscopic opening into the joint cavity in which shaft 1101 passes during the procedure.

In certain embodiments, screen electrode 1102 may comprise a conductive material, such as tungsten, titanium, molybdenum, stainless steel, aluminum, gold, copper or the like. Screen electrode 1102 may have a variety of different shapes and sizes, i.e., comparable to the shapes and sizes of the screen electrode embodiment(s) shown herein in FIGS. 7B and 9. In the present embodiment, screen electrode may comprise a suction opening 1118 (or suction aperture 1118) having sizes and configurations that may vary depending on the particular application. The exposed surface of screen electrode 1102 is preferably generally planar, with no projections extending from the surface of screen electrode 1102 or from an area associated with suction aperture 1118. Suction aperture 1118 will typically be large enough to allow ablated tissue fragments to pass through into a suction/aspiration lumen port and suction/aspiration lumen (not shown) integrated into shaft 1101 of probe 1100.

In configurations according to the present embodiments, suction aperture 1118 is preferably formed in a design that provides for increased aperture edge surface exposure in combination with a sufficient aperture area large enough to allow material desired to be aspirated to enter the suction lumen via the suction lumen port. For example, suction aperture 1118 may preferably be formed in the shape of a star, an asterisk, a lightning bolt, or the like. Consistent with the selected size and shape of the suction opening in screen electrode 1102, suction aperture 1118 is characterized by an aperture perimeter 1119 and an aperture area 1120. In the configurations described in accordance with at least some embodiments, aperture perimeter 1119 may be the sum of the length of the exposed edge surfaces bounding suction aperture 1118, and aperture area 1120 may be the two-dimensional size of the region bounded by the closed aperture perimeter 1119. Alternatively, the aperture area 1120 may be expressed as the total area of the exposed surface of a projected three-dimensional solid corresponding in shape to that of suction aperture 1118.

In comparison and by way of example to further describe the present screen electrode aperture design providing increased edge surface in combination with sufficient area for materials desired to be aspirated to enter the suction lumen, a corresponding and comparative suction aperture 1118′ configured in the shape of a circle having a circular perimeter 1119′ corresponding to an aperture area 1120′ is illustrated in FIG. 11B. Suction aperture 1118′ may be further defined by radius R, such that aperture area 1120′ has a value of π·R² and circular perimeter 1119′ has a value of 2 π·R. Accordingly, the ratio of circular perimeter 1119′ to aperture area 1120′ may be expressed as 2/R.

Referring both to FIGS. 11A and 11B, the present disclosure is directed to designs of active screen electrodes with a single, non-circular aperture for aspirating electrosurgical byproducts. Therefore, in order to provide for such a screen electrode suction aperture design having increased aperture edge surface exposure in combination with a sufficient aperture area large enough to allow materials to enter the suction lumen, in preferred embodiments suction aperture 1118 is configured such that aperture perimeter 1119 has a value substantially greater than a corresponding circular perimeter 1119′ if the related aperture area 1120′ is characterized by a circular shape. Accordingly, it is preferred that the shape of suction aperture 1118 be characterized such that the ratio of aperture perimeter 1119 to aperture area 1120 for at least the useful life of screen electrode 1102 is greater than 2/R as compared to a corresponding suction aperture 1118′ having a generally circular shape with circular perimeter 1119′ with a value of 2 π·R and related to an aperture area 1120′ with a value of π·R².

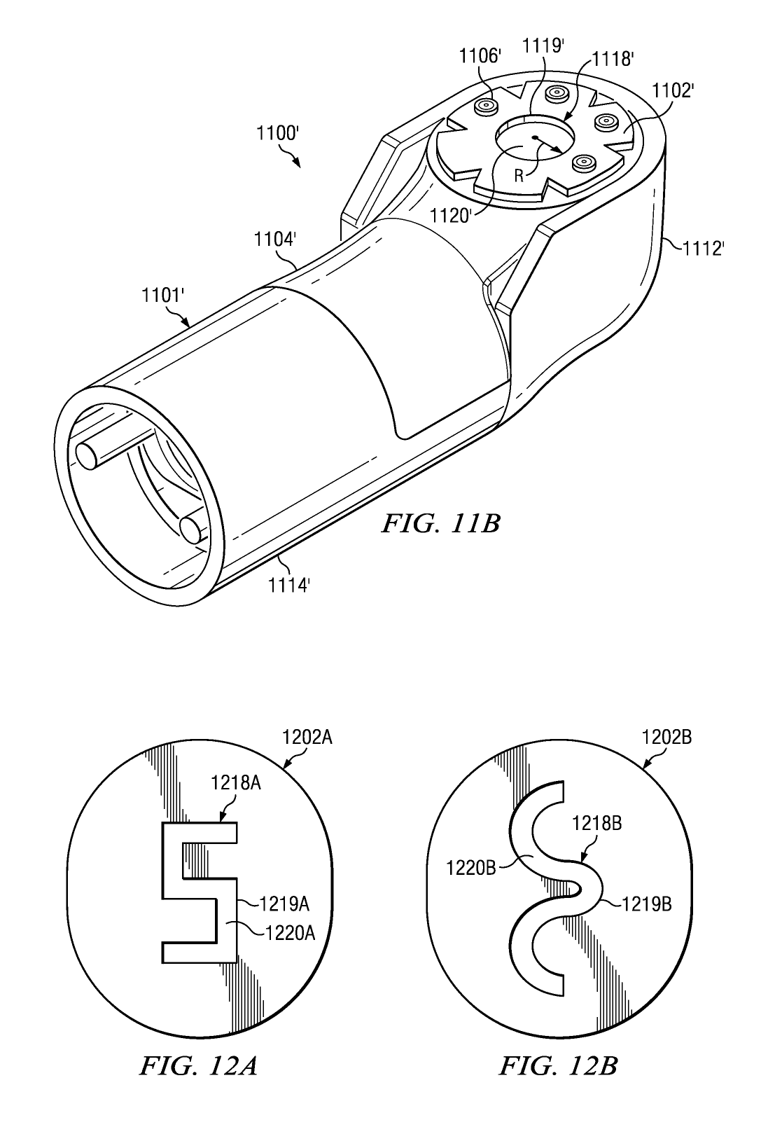

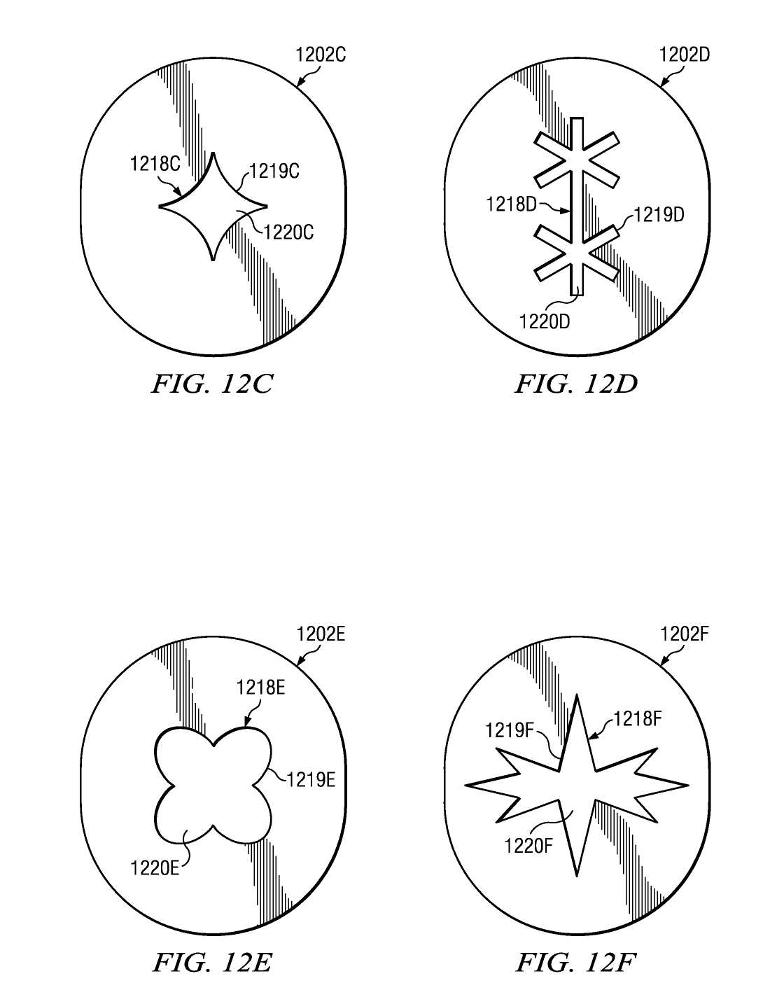

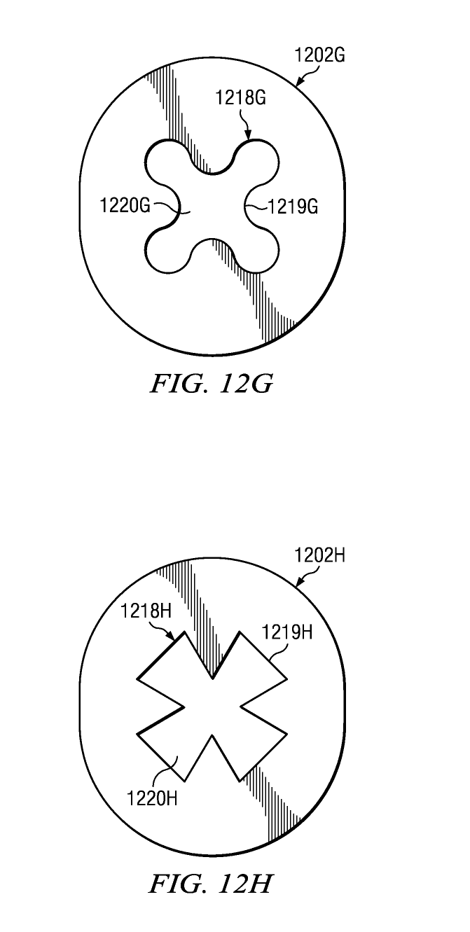

Referring now to FIGS. 12A-H, additional variations of suction aperture configurations are shown by way of example and without limitation to the subject matter of the present claims and disclosure. Designs of suction aperture shapes in accordance with at least some embodiments may have any combination of arcs, angles, projections, or the like defining the exposed edge surfaces of the suction aperture and the aperture perimeter. It is preferred in all embodiments that the exposed screen electrode surface is generally planar, with no projections extending from the surface of screen electrode or from an area associated with the suction aperture. For example, FIG. 12A illustrates screen electrode 1202A having suction aperture 1218A formed in the shape of a “block S.” Suction aperture 1218A may be bounded by an aperture perimeter 1219A that defines an aperture area 1220A. FIG. 12B illustrates screen electrode 1202B having suction aperture 1218B formed in the shape of a “multi-S curve.” Suction aperture 1218B may be bounded by an aperture perimeter 1219B that defines an aperture area 1220B. FIG. 12C illustrates screen electrode 1202C having suction aperture 1218C formed in the shape of a “four-point arched star.” Suction aperture 1218C may be bounded by an aperture perimeter 1219C that defines an aperture area 1220C. FIG. 12D illustrates screen electrode 1202D having suction aperture 1218D formed in the shape of a “double asterisk.” Suction aperture 1218D may be bounded by an aperture perimeter 1219D that defines an aperture area 1220D.

FIG. 12E illustrates screen electrode 1202E having suction aperture 1218E formed in the shape of a “four leaf clover.” Suction aperture 1218E may be bounded by an aperture perimeter 1219E that defines an aperture area 1220E. FIG. 12F illustrates screen electrode 1202F having suction aperture 1218F formed in the shape of a “multi-point star.” Suction aperture 1218F may be bounded by an aperture perimeter 1219F that defines an aperture area 1220F. FIG. 12G illustrates screen electrode 1202G having suction aperture 1218G formed in the shape of “conjoined repeating alternating arcs.” Suction aperture 1218G may be bounded by an aperture perimeter 1219G that defines an aperture area 1220G. FIG. 12H illustrates screen electrode 1202H having suction aperture 1218H formed in the shape of a “block X.” Suction aperture 1218H may be bounded by an aperture perimeter 1219H that defines an aperture area 1220H.

Other modifications and variations can be made to disclose embodiments without departing from the subject invention as defined in the following claims. For example, it should be noted that the invention is not limited to an electrode array comprising a plurality of electrode terminals. The invention could utilize a plurality of return electrodes, e.g., in a bipolar array or the like. In addition, depending on other conditions, such as the peak-to-peak voltage, electrode diameter, etc., a single electrode terminal may be sufficient to contract collagen tissue, ablate tissue, or the like.

In addition, the active and return electrodes may both be located on a distal tissue treatment surface adjacent to each other. The active and return electrodes may be located in active/return electrode pairs, or one or more return electrodes may be located on the distal tip together with a plurality of electrically isolated electrode terminals. The proximal return electrode may or may not be employed in these embodiments. For example, if it is desired to maintain the current flux lines around the distal tip of the probe, the proximal return electrode will not be desired.

While preferred embodiments of this invention have been shown and described, modifications thereof can be made by one skilled in the art without departing from the scope or teaching herein. The embodiments described herein are exemplary only and are not limiting. Because many varying and different embodiments may be made within the scope of the present teachings, including equivalent structures or materials hereafter thought of, and because many modifications may be made in the embodiments herein detailed in accordance with the descriptive requirements of the law, it is to be understood that the details herein are to be interpreted as illustrative and not in a limiting sense.

What is claimed is:

1. An electrosurgical instrument for removing tissue from a target site within or on a patient's body comprising:
a shaft, wherein the shaft has a proximal end and a distal end portion;
an electrode assembly comprising a substantially flat active screen electrode positioned on the distal end portion of the shaft, at least one return electrode positioned on the shaft and spaced away from the active electrode, and at least two securing electrodes positioned on the distal end portion of the shaft and electrically connected to the screen electrode;
an electrically insulating support member upon which the screen electrode is mounted, the support member engaging a portion of the at least one securing electrode for securing the screen electrode; and
wherein the screen electrode comprises an aperture, the aperture has an aperture area and an aperture perimeter, the aperture perimeter substantially greater than a corresponding circular perimeter.

2. The instrument of claim 1, wherein the aperture comprises a star shape.

3. The instrument of claim 1, wherein the aperture comprises an asterisk shape.

4. The instrument of claim 1, wherein the aperture comprises a lightning bolt shape.

5. The instrument of claim 1, wherein the ratio of the aperture perimeter to the aperture area is greater than a ratio of the corresponding circular perimeter to a corresponding circular aperture area.

6. The instrument of claim 1, further comprising an aspiration lumen within the shaft having a distal opening coupled to the screen electrode to inhibit clogging of the lumen and in fluid communication with the aperture.

7. The instrument of claim 1, wherein the screen electrode is brought adjacent a tissue structure immersed in electrically conductive fluid and the electrically conductive fluid completes a conduction path between the screen electrode and the return electrode.

8. The instrument of claim 7, wherein upon the application of a sufficiently high frequency voltage between the screen electrode and the return electrode to vaporize the conductive fluid in a thin layer over at least a portion of the screen electrode to induce the discharge of energy from the vapor layer.

9. The instrument of claim 8, wherein the discharge of energy from the vapor layer is sufficient to form a plasma.

10. The instrument of claim 8, wherein the vapor layer contacts the tissue structure and is capable of ablating a portion of the tissue structure.

11. The instrument of claim 1, further comprising at least one securing electrode positioned on the distal end portion of the shaft and electrically connected to the screen electrode.

12. An electrosurgical instrument for removing tissue from a target site within or on a patient's body comprising:
a shaft, wherein the shaft has proximal and distal end portions;
an active electrode on the distal end portion;
a return electrode on the shaft;
an insulative support body separating the active electrode and the return electrode; and
wherein the active electrode comprises an aperture, the aperture has an aperture area and an aperture perimeter, the aperture perimeter substantially greater than a corresponding circular perimeter.

13. The instrument of claim 12, wherein the aperture comprises a star shape.

14. The instrument of claim 12, wherein the aperture comprises an asterisk shape.

15. The instrument of claim 12, wherein the aperture comprises a lightning bolt shape.

16. The instrument of claim 12, wherein the ratio of the aperture perimeter to the aperture area is greater than a ratio of the corresponding circular perimeter to a corresponding circular aperture area.

17. The instrument of claim 12, further comprising an aspiration lumen within the shaft having a distal opening coupled to the screen electrode to inhibit clogging of the lumen and in fluid communication with the aperture.

18. The instrument of claim 12, wherein the screen electrode is brought adjacent a tissue structure immersed in electrically conductive fluid and the electrically conductive fluid completes a conduction path between the screen electrode and the return electrode.

19. The instrument of claim 18, wherein upon the application of a sufficiently high frequency voltage between the screen electrode and the return electrode to vaporize the conductive fluid in a thin layer over at least a portion of the screen electrode to induce the discharge of energy from the vapor layer.

20. The instrument of claim 19, wherein the discharge of energy from the vapor layer is sufficient to form a plasma.

21. The instrument of claim 19, wherein the vapor layer contacts the tissue structure and is capable of ablating a portion of the tissue structure.

22. The instrument of claim 12, further comprising at least one securing electrode electrically coupled to the active electrode and securing the active electrode to the shaft.
